(19) United States
(12) Patent Application Publication
(10) Pub. No.: US 2006/0286469 A1
(43) Pub. Date: Dec. 21, 2006

(54) Imaging members

(75) Inventors: Jin Wu, Webster, NY (US); Justin F. Galloway, Webster, NY (US); Liang-Bih Lin, Rochester, NY (US)

Correspondence Address: Carter, DeLuca, Farrell & Schmidt, LLP;Suite 225, 445 Broad Hollow Road, Melville, NY, 11747, US

(73) Assignee: Xerox Corporation

(21) Appl. No.: 11/154,449

(22) Filed: Jun. 16, 2005

(51) Int. Cl. G03G 5/06; C07D 487/22

(52) U.S. Cl. 430/58.8; 430/78; 430/59.4; 540/141; 540/123; 540/136

(57) ABSTRACT

Methods for preparing phthalocyanine pigments having a high surface area are provided.

### BACKGROUND

The present disclosure relates to phthalocyanine dyes or pigments for use in photoreceptors and, more particularly, to methods for the production of hydroxygallium phthalocyanines.

Hydroxygallium phthalocyanine (HOGaPc) pigments are currently utilized in photoreceptors as photogenerating components. HOGaPc polymorphs are known, including the Type V polymorph. U.S. Pat. Nos. 5,521,306 and 5,473,064, the entire disclosures of each of which are incorporated by reference herein, describe HOGaPc and processes to prepare the Type V polymorph of HOGaPc. The Type V polymorph has been characterized by its intense diffraction peaks at Bragg angles 7.5, 9.9, 12.5, 16.3, 18.6, 21.9, 23.9, 25.1, and 28.3, with the highest peak at 7.5 degrees 2Θ (2 theta±0.2°) in the X-ray diffraction spectrum.

HOGaPc is responsive at a range of, for example, about 550 nanometers to about 880 nanometers and is generally unresponsive to the light spectrum below about 500 nanometers. Wavelengths for photogeneration are from about 600 nanometers to about 850 nanometers and may include a broadband between the two wavelengths. Single wavelength exposure is frequently from about 750 nanometers to about 850 nanometers.

Known HOGaPc pigments possess surface areas of about 40 m2/g. However, large HOGaPc pigment particles present in a photogenerating layer may cause print defects including charge deficient spots, which may result in poor image quality.

Therefore, HOGaPc pigments made of smaller particles, and thus possessing a larger surface area, are desirable. Raw pigments having a higher surface area result in charge generation layers with pigments having finer particle sizes, and thus higher surface area, which may extend the useful life of photoreceptors possessing such fine pigments by reducing the formation of charge deficient spots.

### SUMMARY

The present disclosure provides processes including contacting a gallium phthalocyanine in an acid solution with a solvent system comprising water, at least one base, and at least one water miscible solvent.

In embodiments, the process includes contacting an alkoxy-bridged gallium phthalocyanine dimer in an acid solution with a solvent system comprising water, aqueous ammonia solution, and at least one water miscible solvent to obtain a Type I hydroxygallium phthalocyanine. The resulting Type I hydroxygallium phthalocyanine may then be converted to a Type V hydroxygallium phthalocyanine. Particles of the resulting Type V hydroxygallium phthalocyanine may have a surface area of from about 50 m2/g to about 100 m2/g. In embodiments, the at least one water miscible solvent may be a cyclic ether, an amide, a polyol, a nitrile, a sulfur-containing solvent, and/or a mixture thereof.

Photoreceptors having photogenerating layers possessing such hydroxygallium phthalocyanines are also provided. In embodiments, the photoreceptor may include a photogenerating layer which, in turn, includes a resin and a photogenerating component. The photogenerating component may include a Type V hydroxygallium phthalocyanine prepared by contacting a gallium phthalocyanine in an acid solution with a solvent system to obtain a Type I hydroxygallium phthalocyanine. The solvent system may include water, aqueous ammonia solution, and at least one water miscible solvent. The Type I hydroxygallium phthalocyanine may then be converted to a Type V hydroxygallium phthalocyanine.

### BRIEF DESCRIPTION OF THE DRAWINGS

Various embodiments of the present disclosure will be described herein below with reference to the figures wherein:

FIGS. 1A, 1B, 1C and 1D are X-ray diffraction data comparing known Type I and Type V HOGaPc polymorphs with high surface area Type I and Type V HOGaPc polymorphs prepared in accordance with the present disclosure.

### EMBODIMENTS

The present disclosure provides processes for the preparation of hydroxygallium phthalocyanine, especially the Type V polymorph, resulting in particles having a high surface area. Type V HOGaPc particles having a high surface area refers, in embodiments, for example to particles having a surface area of from about 50 m2/g to about 100 m2/g, in embodiments from about 60 m2/g to about 80 m2/g. Photoresponsive imaging members utilizing such high surface area photogenerating components are also provided.

The process of the present disclosure may be utilized to first obtain a high-surface area Type I HOGaPc. High surface area Type I HOGaPc refers, in embodiments, for example to Type I HOGaPc having a surface area from about 50 m2/g to about 90 m2/g, in embodiments from about 60 m2/g to about 70 m2/g. In embodiments, the process of the present disclosure includes the preparation of a chlorogallium phthalocyanine, the hydrolysis of the chlorogallium phthalocyanine to hydroxygallium phthalocyanine by dissolving the chlorogallium phthalocyanine in a concentrated acid, and then adding the resulting phthalocyanine solution to a solvent system of the present disclosure to precipitate high surface area hydroxygallium phthalocyanine Type I.

For example, a suitable process may include the preparation of hydroxygallium phthalocyanine, essentially free of a halide, like chlorine, whereby a pigment precursor Type I halogallium phthalocyanine, in embodiments a chlorogallium phthalocyanine, is prepared by the reaction of gallium chloride in a solvent, such as N-methylpyrrolidone, present in an amount of from about 10 parts to about 100 parts, in embodiments about 15 to about 25 parts, and in embodiments about 19 parts, with 1,3-diiminoisoindoline in an amount of from about 1 part to about 10 parts, in embodiments about 2 parts to about 6 parts of 1,3-diiminoisoindoline, for each part of gallium chloride that is reacted. The pigment precursor chlorogallium phthalocyanine Type I is then hydrolyzed by standard methods, for example acid pasting, whereby the pigment precursor is dissolved in a concentrated acid such as sulfinuric acid, hydrogen halides including hydrochloric acid (HCL), hydrobromic acid (HBr), hydroiodic acid (HI), oxyacids of halogens including chloric acid (HClO3), perchloric acid (HClO4), bromic acid (HBrO3), perbromic acid (HBrO4), iodic acid (HIO3), periodic acid (HIO4), nitric acid, and/or trifluoroacetic acid, to form a solution. The chlorogallium phthalocyanine pigment can be dissolved in the concentrated acid in an amount of from about 1 weight part to about 100 weight parts, in embodiments from about 25 weight parts to about 75 weight parts, by stirring said pigment in the acid for an effective period of time, from about one minute to about 24 hours, in embodiments from about 2 hours to about 4 hours. The temperature of the solution can be from about 0° C. to about 80° C., in embodiments from about 40° C. to about 60° C., in air or under an inert atmosphere such as argon or nitrogen. The resulting mixture may be filtered through a 5-μm glass filter to remove any insoluble pigments. The pigment precursor may then be reprecipitated in a solvent system of the present disclosure to obtain a high surface area Type I hydroxygallium phthalocyanine.

In embodiments, the process of the present disclosure also includes a high surface area Type I hydroxygallium phthalocyanine prepared by the hydrolysis of a dimer. The process includes the preparation of an alkoxy-bridged gallium phthalocyanine dimer, the hydrolysis of the phthalocyanine dimer to hydroxygallium phthalocyanine by dissolving the dimer in a concentrated acid, and then adding the resulting phthalocyanine solution to a solvent system of the present disclosure to precipitate high surface area hydroxygallium phthalocyanine Type I.

Embodiments of the present disclosure thus include the dissolution of 1 part gallium chloride in about 1 part to about 100 parts, in embodiments about 5 parts to about 15 parts, of an organic solvent. Suitable organic solvents include, for example, aromatics including benzene, toluene, xylene and the like. The reaction can occur at a temperature of from about 0° C. to about 100° C., in embodiments at a temperature of from about 20° C. to about 30° C., to form a solution of gallium chloride. The gallium chloride solution is contacted with from about 1 part to about 5 parts, in embodiments from about 2 parts to about 4 parts, of an alkali metal alkoxide such as sodium methoxide, sodium ethoxide, sodium propoxide or the like, in embodiments in a solution form, to produce a gallium alkoxide solution and an alkali metal salt byproduct, for example sodium chloride. The reaction can occur at a temperature of from about 0° C. to about 100° C., in embodiments at a temperature of from about 20° C. to about 40° C.

The alkali metal salt byproduct may be removed from the resulting gallium alkoxide solution by reaction with from about 1 part to about 10 parts, in embodiments from about 2 parts to about 6 parts, orthophthalodinitrile or 1,3-diiminoisoindolene, and a diol, such as 1,2-ethanediol (ethylene glycol), 1,2-propanediol (propylene glycol) or 1,3-propanediol, in an amount of from about 3 parts to about 100 parts, in embodiments from about 5 parts to about 15 parts, for each part of gallium alkoxide formed. The reaction can occur at a temperature of from about 150° C. to about 220° C., in embodiments at a temperature of from about 185° C. to about 205° C., for a period of about 30 minutes to about 6 hours, in embodiments about 1 hour to about 3 hours, to provide an alkoxy-bridged gallium phthalocyanine dimer pigment precursor. This dimer pigment may be isolated by filtration at a temperature of from about 20° C. to about 180° C., in embodiments from about 100° C. to about 140° C.

The dimer precursor may then be added to a concentrated acid, such as sulfuric acid, hydrogen halides including hydrochloric acid (HCl), hydrobromic acid (IBr), hydroiodic acid (HI), oxyacids of halogens including chloric acid (HClO3), perchloric acid (HClO4), bromic acid (HBrO3), perbromic acid (HBrO4), iodic acid (HIO3), periodic acid (HIO4), nitric acid, and/or trifluoroacetic acid, to form a solution. Halogenated organic solvents may be added to dissolve the dimer, wherein the volume/volume ratio of the acid to the halogenated solvent is from about 1/10 to about 10/1, in embodiments from about 1/1 to about 5/1. Examples of halogenated solvents include methylene chloride, chloroform, 1,2-dichloroethane, 1,1,2-tricloroethane, and monochlorobenzene. The alkoxy-bridged gallium phthalocyanine dimer pigment can be dissolved in the concentrated acid in an amount of from about 1 weight part to about 100 weight parts, in embodiments from about 25 weight parts to about 75 weight parts, by stirring said pigment in the acid for an effective period of time, from about one minute to about 24 hours, in embodiments from about 2 hours to about 4 hours. The temperature of the solution can be from about 0° C. to about 80° C., in embodiments from about 40° C. to about 60° C., in air or under an inert atmosphere such as argon or nitrogen. The resulting mixture may be filtered through a 5-μm glass filter to remove any insoluble pigments.

The resulting mixture, whether obtained by hydrolysis of chlorogallium phthalocyanine or hydrolysis of an alkoxy-bridged gallium phthalocyanine dimer, is added at a controlled rate to a solvent system of the present disclosure in what may be referred to, in embodiments, as an acid pasting step. The solvent system may include, for example, water, a base, and a water miscible solvent. In embodiments the water miscible solvent is better than water as a solvent for the HOGaPc pigment.

Suitable bases which can be used include hydroxides, amines, and the like. Specific examples include an aqueous ammonia solution, ammonia, trimethylammonia, pyridine, hydrazine, hydroxylamine, methylamine, ethylamine, dimethylamine, diethylamine, ethanolamine, triethanolamine, ethyldiamine, urea, aqueous hydrogen bisulfide (HS—) solution, and conjugated bases of weak acids such as aqueous formate (HCOO—) solution. The base amount is added into the pasting solvent system according to the acid amount in the resulting pigment mixture. Suitable bases have a pKb≧1. In embodiments, the pKb of the base can be from about 1 to about 10, in embodiments from about 3 to about 5. The volume percentage of the base in the pasting solvent system is from about 10 to about 70, in embodiments from about 30 to about 50. The molar ratio of the resulting base/acid pair is from about 2/1 to about 1/2, in embodiments from about 1.2/1 to about 1/1.2. At least one base may be utilized; in embodiments from about 2 to about 5 bases may be utilized.

Suitable water miscible solvents which may be utilized include cyclic ethers, amides, polyols, nitrites, and sulfur containing solvents. Examples of water miscible solvents include tetrahydrofuran (THF), N,N-dimethylformamide (DMF), N,N-dimethylacetamide (DMAc), dimethyl sulfoxide (DMSO), tetramethylene sulfone, acetonitrile, 1,3-dioxane, 1,4-dioxane, ethylene glycol, 1-methyl-2-pyrrolidinone, and combinations thereof. The volume percentage of water miscible solvent added to the pasting solvent system can be from about 5 to about 70 of the pasting solvent system, in embodiments from about 20 to about 40 of the pasting solvent system. At least one water miscible solvent may be utilized; in embodiments from about 2 to about 5 water miscible solvents may be utilized.

In embodiments, the amount of the water, base, and water miscible solvent in the solvent system totals about 100 percent. In embodiments, the solvents utilized include a combination of ammonia, deionized water, and at least one water miscible solvent such as THF, DMF, and the like.

In embodiments, the use of water miscible solvents in the solvent system result in pigments which exhibit finer and more uniform morphology when compared with those formed in non-solvents without the water miscible solvent. Without wishing to be bound by any theory, one reason for the finer and more uniform morphology may be because the crystallization process is slower with the water miscible solvent and smaller crystals are formed.

The combined solvent system may be chilled while being stirred during pigment precipitation in order to maintain a temperature of from about −20° C. to about 40° C., in embodiments from about 0° C. to about 10° C., during pigment precipitation. The resulting pigment may be isolated by, for example, filtration or centrifugation. In embodiments, the filtrate may be washed with deionized water to obtain a filtrate of a neutral pH.

The resulting high surface area hydroxygallium phthalocyanine possesses X-ray diffraction patterns having major peaks at Bragg angles of 7.0°, 13.4°, 15.7°, 16.9°, 26.0°, 26.8° 2Θ (2 theta±0.2°), referred to, in embodiments, as high surface area Type I hydroxygallium phthalocyanine.

The surface area of the high surface area Type I hydroxygallium phthalocyanine is from about 50 m2/g to about 90 m2/g, in embodiments from about 60 m2/g to about 70 m2/g.

Methods which may be utilized to determine the surface area of the HOGaPc include, for example, the Brunauer, Emmett and Teller (BET) method or mercury porosimetry. In embodiments, a multi point BET method using nitrogen as the adsorbate may be utilized.

In the BET process, about 0.5 grams of a sample may be weighed into analysis vessels. The samples may be degassed at about 50° C. under full vacuum overnight, in embodiments about 12 to about 20 hours, prior to analysis. The surface area may be determined using nitrogen as the adsorbate gas at 77 Kelvin (LN2), over a relative pressure range of about 0.08 to about 0.25 using a Micromeritics ASAP 2405 surface area instrument. The surface area is the BET surface area minus the micropore area. Micropores are pores with a diameter of 20 angstroms or less. Micropore areas may be calculated for the HOGaPc pigments using the Harkins and Jura method over the specified thickness range of 6 to 10 angstroms. The surface area obtained by this method is comparable to the surface area as determined by mercury porosimetry.

The high surface area Type I hydroxygallium phthalocyanine obtained by the process of the present disclosure is then converted to high surface area Type V hydroxygallium phthalocyanine. The Type I hydroxygallium phthalocyanine product obtained can be converted to Type V hydroxygallium phthalocyanine by contacting the Type I HOGaPc with a polar aprotic solvent, such as N,N-dimethylformamide, N-methylpyrrolidone, or the like by, for example, stirring, ball milling or otherwise contacting said Type I hydroxygallium phthalocyanine pigment with the aforementioned solvent in the absence or presence of grinding media such as stainless steel shot, spherical or cylindrical ceramic media, or spherical glass beads. Ketones may be added as a second solvent in the presence of the polar aprotic solvent. Examples of ketones include acetone, methyl ethyl ketone, methyl isobutyl ketone. The volume ratio of the ketone to the aprotic solvent can be from about 30/70 to about 70/30, in embodiments from about 40/60 to about 60/40. The Type I HOGaPc product may be combined with the solvent and grinding media at a temperature of from about 0° C. to about 40° C., in embodiments from about 10° C. to about 30° C., for a period of from about 2 hours to about 2 weeks, in embodiments from about 72 hours to about 1 week, with constant rolling speed from about 30 rpm to about 150 rpm, in embodiments from about 50 rpm to about 70 rpm, such that there is obtained a Type V hydroxygallium phthalocyanine polymorph.

In embodiments, the Type I HOGaPc may be placed in DMF and combined with glass beads for milling. Suitable glass beads include, for example, from about 1 mm to about 6 mm soda lime glass beads (Glen Mills, Inc., Clifton, N.J.), and borosilicate glass beads (Hi-Bea D20 from Ohara Inc., Kanagawa, Japan). About 2 grams to about 100 grams of Type I HOGaPc, in embodiments from about 5 grams to about 7 grams of the Type I HOGaPc, may be contacted with about 16 grams to about 800 grams DMF, in embodiments about 40 grams to about 80 grams DMF, with about 60 grams to about 3000 grams of beads, in embodiments from about 160 grams to about 200 grams of beads, and milled for about 2 hours to about 2 weeks, in embodiments from about 72 hours to about 1 week, with constant rolling speeds from about 30 rpm to about 150 rpm, in embodiments from about 50 rpm to about 70 rpm.

The resulting high surface area Type V HOGaPc may be separated from the mixture by washing with DMF, acetone, or combinations thereof followed by filtration. The Type V polymorph may then be dried under vacuum at a temperature of from about 50° C. to about 95° C., in embodiments from about 65° C. to about 80° C. and then crushed utilizing ball milling, rotary milling, and the like.

The resulting high surface area HOGaPc possesses an X-ray diffraction pattern having major peaks at Bragg angles of 7.5°, 9.9°, 12.5°, 16.3°, 18.6°, 21.9°, 23.9°, 25.1°, and 28.3°, with the highest peak at 7.5°2Θ(2 theta±0.2°), referred to, in embodiments, as high surface area Type V hydroxygallium phthalocyanine.

The surface area of the high surface area Type V hydroxygallium phthalocyanine can be from about 50 m2/g to about 100 m2/g, and in embodiments from about 60 m2/g to about 80 m2/g. The surface area of the high surface area Type V polymorph does not change much from the surface area of the high surface area Type I polymorph because the subsequent conversion of the Type I polymorph to the Type V polymorph mostly changes the surface properties of the crystal, not the crystal size. The processes of the present disclosure thus avoid difficulties associated with grinding down dense and large Type I pigments that may form in the acid-pasting step of previously utilized processes. Such difficulties include, for example, waste of pigment, obtaining uniform and smaller size of pigment particles.

The high surface area Type V hydroxygallium phthalocyanine obtained according to the present disclosure exhibits excellent properties in photoresponsive imaging members when used as a pigment, in particular, lower print background, lower charge deficient spots (CDS), lower dark decay and better cyclic stability compared to low surface area Type V hydroxygallium phthalocyanine obtained via previously utilized processes, for example, from dimer or other gallium phthalocyanine precursors such as, for example, chlorogallium phthalocyanine.

The hydroxygallium phthalocyanine pigments produced in accordance with the present disclosure may be utilized as a photogenerating component and combined with a resin to form a photogenerating layer of a photoreceptor. Examples of suitable resins for use in preparing the dispersion include thermoplastic and thermosetting resins such as polycarbonates, polyesters including poly(ethylene terephthalate), polyurethanes including poly(tetramethylene hexamethylene diurethane), polystyrenes including poly(styrene-co-maleic anhydride), polybutadienes including polybutadiene-graft-poly(methyl acrylate-co-acrylontrile), polysulfones including poly(1,4-cyclohexane sulfone), polyarylethers including poly(phenylene oxide), polyarylsulfones including poly(phenylene sulfone), polyethersulfones including poly (phenylene oxide-co-phenylene sulfone), polyethylenes including poly(ethylene-co-acrylic acid), polypropylenes, polymethylpentenes, polyphenylene sulfides, polyvinyl acetates, polyvinylbutyrals, polysiloxanes including poly(dimethylsiloxane), polyacrylates including poly (ethyl acrylate), polyvinyl acetals, polyamides including poly(hexamethylene adipamide), polyimides including poly(pyromellitimide), amino resins including poly(vinyl amine), phenylene oxide resins including poly(2,6-dimethyl-1,4-phenylene oxide), terephthalic acid resins, phenoxy resins including poly(hydroxyethers), epoxy resins including poly([(o-cresyl glycidyl ether)-co-formaldehyde], phenolic resins including poly(4-tert-butylphenol-co-formaldehyde), polystyrene and acrylonitrile copolymers, polyvinylchlorides, polyvinyl alcohols, poly-N-vinylpyrrolidinones, vinylchloride and vinyl acetate copolymers, carboxyl-modified vinyl chloride/vinyl acetate copolymers, hydroxyl-modified vinyl chloride/vinyl acetate copolymers, carboxyl- and hydroxyl-modified vinyl chloride/vinyl acetate copolymers, acrylate copolymers, alkyd resins, cellulosic film formers, poly(amideimide), styrene-butadiene copolymers, vinylidenechloride-vinylchloride copolymers, vinylacetate-vinylidenechloride copolymers, styrene-alkyd resins, polyvinylcarbazoles, and the like, and combinations thereof. These polymers may be block, random, or alternating copolymers.

Examples of suitable polycarbonates which may be utilized to form the photogenerating layer dispersion include, but are not limited to, poly(4,4′-isopropylidene diphenyl carbonate) (also referred to as bisphenol A polycarbonate), poly(4,4′-diphenyl-1,1′-cyclohexane carbonate) (also referred to as bisphenol Z polycarbonate, polycarbonate Z, or PCZ), poly(4,4′-sulfonyl diphenyl carbonate) (also referred to as bisphenol S polycarbonate), poly(4,4′-ethylidene diphenyl carbonate) (also referred to as bisphenol E polycarbonate), poly (4,4′-methylidene diphenyl carbonate) (also referred to as bisphenol F polycarbonate), poly (4,4′-(1,3-phenylenediisopropylidene)diphenyl carbonate) (also referred to as bisphenol M polycarbonate), poly(4,4′-(1,4-phenylenediisopropylidene)diphenyl carbonate) (also referred to as bisphenol P polycarbonate), poly(4,4′-hexafluoroisppropylidene diphenyl carbonate).

Examples of suitable vinyl chloride and vinyl acetate copolymers which may be utilized to form the dispersion utilized to form the photogenerating layer include, but are not limited to, carboxyl-modified vinyl chloride/vinyl acetate copolymers such as VMCH (available from Dow Chemical), hydroxyl-modified vinyl chloride/vinyl acetate copolymers such as VAGF (available from Dow Chemical), and carboxyl/hydroxyl-modified vinyl chloride/vinyl acetate copolymers such as UCARMAG® 527 (available from Dow Chemical).

The molecular weight of the resin used to form the photogenerating layer may be from about 10,000 to about 100,000, in embodiments from about 15,000 to about 50,000.

In embodiments, a single resin may be utilized to form the photogenerating layer. In other embodiments, a mixture of more than one of the above resins can be used to form the photogenerating layer. Where more than one resin is utilized, the number of resins can be from about 2 to about 4, in embodiments from about 2 to about 3.

A liquid or liquid mixture may be used in preparing the photogenerating layer. A liquid mixture may include from about 2 to about 4 liquids, in embodiments from about 2 to about 3 liquids. In embodiments, the liquid is a solvent for the resin, but not the high surface area Type V HOGaPc of the present disclosure. The resin may be added to the liquid, in embodiments a solvent for the resin, to form a solution and the pigment then added to the solution to form a dispersion suitable for forming the photogenerating layer. The liquid utilized should not substantially disturb or adversely affect other layers of the photoreceptor, if any. Examples of liquids that can be utilized in preparing the photogenerating layer include, but are not limited to, ketones, alcohols, aromatic hydrocarbons, halogenated aliphatic hydrocarbons, ethers, amines, amides, esters, mixtures thereof, and the like. Specific illustrative examples include cyclohexanone, acetone, methyl ethyl ketone, methanol, ethanol, butanol, amyl alcohol, toluene, xylene, monochlorobenzene, carbon tetrachloride, chloroform, methylene chloride, trichloroethylene, tetrahydrofuran, dioxane, diethyl ether, dimethyl formamide, dimethyl acetamide, n-butyl acetate, ethyl acetate, methoxyethyl acetate, mixtures thereof, and the like.

The resin in a liquid, which is a solvent for the resin, is combined with the high surface area Type V HOGaPc of the present disclosure. Any suitable technique may be utilized to disperse the high surface area Type V HOGaPc in the resin or resins. The dispersion containing the pigment may be formed using, for example, attritors, ball mills, Dynomills, paint shakers, homogenizers, microfluidizers, mechanical stirrers, in-line mixers, ultrasonic processor, Cavipro processor, or by any other suitable milling techniques.

Specific dispersion techniques which may be utilized include, for example, ball milling, roll milling, milling in vertical or horizontal attritors, sand milling, and the like. The solids content of the mixture being milled can be selected from a wide range of concentrations. Milling times using a ball roll mill may be between about 6 hours and about 6 days, in embodiments from about 8 hours to about 3 days. However, as noted above, in embodiments milling of large particles is not required as the methods of the present disclosure result in high surface area Type V HOGaPc.

The amount of resin in the dispersion can be from about 95% by weight to about 15% by weight of the solids, in embodiments from about 65% by weight to about 35% by weight of the solids. The amount of pigment in the dispersion can be from about 5% by weight to about 85% by weight of the dispersion solids, in embodiments from about 35% by weight to about 65% by weight of the dispersion solids. The expression “solids” refers to the total pigment and resin components of the dispersion.

Any suitable and conventional technique may be utilized to apply the dispersion of the present disclosure to form a photogenerating layer on another layer of a photoreceptor. Suitable coating techniques include dip coating, roll coating, spray coating, rotary atomizers, and the like.

The photogenerating layer containing the pigments of the present disclosure as a photogenerating component and the resinous material may be of a thickness from about 0.05 μm to about 5 μm, in embodiments from about 0.1 μm to about 1 μm, although the thickness can be outside these ranges. The photogenerating layer thickness is related to the relative amounts of pigment and resin, with the pigment often being present in amounts from about 5 to about 85 percent by weight, in embodiments from about 35 to about 65 percent by weight. Higher resin content compositions generally require thicker layers for photogeneration. Generally, it may be desirable to provide this layer in a thickness sufficient to absorb about 90 percent or more of the incident radiation which is directed upon it in the imagewise or printing exposure step. The maximum thickness of this layer depends upon factors such as mechanical considerations, the thicknesses of the other layers, and whether a flexible photoconductive imaging member is desired.

The dispersions of the present disclosure may be utilized to form photogenerating layers in conjunction with any known configuration for photoreceptors, including single and multi-layer photoreceptors. Examples of multi-layer photoreceptors include those described in U.S. Pat. Nos. 6,800,411, 6,824,940, 6,818,366, 6,790,573, and U.S. Patent Application Publication No. 20040115546, the entire disclosures of each of which are incorporated by reference herein. Photoreceptors may possess a photogenerating layer (CGL), also referred to herein as a photogenerating layer, and a charge transport layer (CTL). Other layers, including a substrate, an electrically conductive layer, a charge blocking or hole blocking layer, an adhesive layer, and/or an overcoat layer, may also be present in the photoreceptor.

Suitable substrates which may be utilized in forming a photoreceptor include opaque or substantially transparent substrates, and may include any suitable organic or inorganic material having the requisite mechanical properties.

The substrate may be flexible, seamless, or rigid and may be of a number of different configurations such as, for example, a plate, a cylindrical drum, a scroll, an endless flexible belt, a web, and the like.

The thickness of the substrate layer may depend on numerous factors, including mechanical performance and economic considerations. For rigid substrates, the thickness of the substrate can be from about 0.3 millimeters to about 10 millimeters, in embodiments from about 0.5 millimeters to about 5 millimeters. For flexible substrates, the substrate thickness can be from about 65 to about 200 micrometers, in embodiments from about 75 to about 100 micrometers, for optimum flexibility and minimum stretch when cycled around small diameter rollers of, for example, 19-millimeter diameter. The entire substrate can be made of an electrically conductive material, or the electrically conductive material can be a coating on a polymeric substrate.

Substrate layers selected for the imaging members of the present disclosure, and which substrates can be opaque or substantially transparent, may include a layer of insulating material including inorganic or organic polymeric materials such as MYLAR® (a commercially available polymer from DuPont), MYLAR® containing titanium, a layer of an organic or inorganic material having a semiconductive surface layer, such as indium tin oxide or aluminum arranged thereon, or a conductive material inclusive of aluminum, chromium, nickel, brass, or the like.

Any suitable electrically conductive material can be employed with the substrate. Suitable electrically conductive materials include copper, brass, nickel, zinc, chromium, stainless steel, conductive plastics and rubbers, aluminum, semi-transparent aluminum, steel, cadmium, silver, gold, zirconium, niobium, tantalum, vanadium, hafnium, titanium, nickel, chromium, tungsten, molybdenum, paper rendered conductive by the inclusion of a suitable material therein, or through conditioning in a humid atmosphere to ensure the presence of sufficient water content to render the material conductive, indium, tin, metal oxides, including tin oxide and indium tin oxide, and the like.

After formation of an electrically conductive surface, a hole blocking layer may optionally be applied to the substrate layer. Generally, hole blocking layers (also referred to, in embodiments, as charge blocking layers, or undercoat layers) allow electrons from the conductive layer to migrate toward the photogenerating layer. Any suitable blocking layer capable of forming an electronic barrier to holes between the adjacent photogenerating layer and the underlying conductive layer of the substrate may be utilized. Suitable blocking layers include those disclosed, for example, in U.S. Pat. Nos. 4,286,033, 4,291,110 and 4,338,387, the entire disclosures of each of which are incorporated herein by reference. Similarly, illustrated in U.S. Pat. Nos. 6,255,027, 6,177,219, and 6,156,468, the entire disclosures of each of which are incorporated herein by reference, are, for example, photoreceptors containing a hole blocking layer of a plurality of light scattering particles dispersed in a binder. For example, Example 1 of U.S. Pat. No. 6,156,468 discloses a hole blocking layer of titanium dioxide dispersed in a linear phenolic binder.

Hole blocking layers utilized for negatively charged photoreceptors may include, for example, polyamides including LUCKAMIDE® (a nylon type material derived from methoxymethyl-substituted polyamide commercially available from Dai Nippon Ink), hydroxy alkyl methacrylates, nylons, gelatin, hydroxyl alkyl cellulose, organopolyphosphazines, organosilanes, organotitanates, organozirconates, metal oxides of titanium, chromium, zinc, tin, silicon, and the like. In embodiments the hole blocking layer may include nitrogen containing siloxanes. Nitrogen containing siloxanes may be prepared from coating solutions containing a hydrolyzed silane. Hydrolyzable silanes include 3-aminopropyl triethoxy silane, N,N′-dimethyl 3-amino propyl triethoxysilane, N,N-dimethylamino phenyl triethoxy silane, N-phenyl aminopropyl trimethoxy silane, trimethoxy silylpropyldiethylene triamine and mixtures thereof.

In embodiments, the hole blocking components may be combined with phenolic compounds, a phenolic resin, or a mixture of more than one phenolic resin, for example, from about 2 to about 4 phenolic resins. Suitable phenolic compounds which may be utilized may contain at least two phenol groups, such as bisphenol A (4,4′-isopropylidenediphenol), bisphenol E (4,4′-ethylidenebisphenol), bisphenol F (bis(4-hydroxyphenyl)methane), bisphenol M (4,4′-(1,3-phenylenediisopropylidene)bisphenol), bisphenol P (4,4′-(1,4-phenylene diisopropylidene)bisphenol), bisphenol S (4,4′-sulfonyldiphenol), and bisphenol Z (4,4′-cyclohexylidenebisphenol), hexafluorobisphenol A (4,4′-(hexafluoro isopropylidene)diphenol), resorcinol, hydroxyquinone, catechin, and the like.

The hole blocking layer may be applied as a coating on a substrate or electrically conductive layer by any suitable conventional technique such as spraying, die coating, dip coating, draw bar coating, gravure coating, silk screening, air knife coating, reverse roll coating, vacuum deposition, chemical treatment, and the like. For convenience in obtaining thin layers, the blocking layers may be applied in the form of a dilute solution, with the solvent being removed after deposition of the coating by conventional techniques such as by vacuum, heating and the like. Drying of the deposited coating may be effected by any suitable conventional technique such as oven drying, infrared radiation drying, air drying and the like.

The blocking layer may be continuous and have a thickness of from about 0.01 micrometers to about 30 micrometers, in embodiments from about 0.1 micrometers to about 20 micrometers.

An optional adhesive layer may be applied to the hole blocking layer. Any suitable adhesive layer known in the art may be utilized including, but not limited to, polyesters, polyamides, poly(vinyl butyral), poly(vinyl alcohol), polyurethane and polyacrylonitrile. Where present, the adhesive layer may be, for example, of a thickness of from about 0.001 micrometers to about 2 micrometers, in embodiments from about 0.01 micrometers to about 1 micrometer. Optionally, the adhesive layer may contain effective suitable amounts, for example from about 1 weight percent to about 10 weight percent, of conductive and nonconductive particles, such as zinc oxide, titanium dioxide, silicon nitride, carbon black, and the like, to provide further desirable electrical and optical properties to the photoreceptor of the present disclosure. Conventional techniques for applying an adhesive layer coating mixture to the hole blocking layer include spraying, dip coating, roll coating, wire wound rod coating, gravure coating, die coating and the like. Drying of the deposited coating may be effected by any suitable conventional technique such as oven drying, infrared radiation drying, air drying and the like.

In embodiments the photoreceptor also includes a charge transport layer attached to the photogenerating layer. The charge transport layer may include a charge transport or hole transport molecule (HTM) dispersed in an inactive polymeric material. These compounds may be added to polymeric materials which are otherwise incapable of supporting the injection of photogenerated holes from the photogenerating layer and incapable of allowing the transport of these holes therethrough. The addition of these HTMs converts the electrically inactive polymeric material to a material capable of supporting the direction of photogenerated holes from the photogenerating layer and capable of allowing the transport of these holes through the charge transport layer in order to discharge the surface charge on the charge transport layer.

Suitable polymers for use in forming the charge transport layer are known film forming resins. Examples include those polymers utilized to form the photogenerating layer. In embodiments resin materials for use in forming the charge transport layer are electrically inactive resins including polycarbonate resins having a weight average molecular weight from about 20,000 to about 150,000, in embodiments from about 50,000 about 120,000. Electrically inactive resin materials which may be utilized in the charge transport layer include poly(4,4′-dipropylidene-diphenylene carbonate) with a weight average molecular weight of from about 35,000 to about 40,000, available as LEXAN® 145 from General Electric Company; poly(4,4′-propylidene-diphenylene carbonate) with a weight average molecular weight of from about 40,000 to about 45,000, available as LEXAN® 141 from the General Electric Company; a polycarbonate resin having a weight average molecular weight of from about 50,000 to about 100,000, available as MAKROLON® from Farbenfabricken Bayer A. G.; a polycarbonate resin having a weight average molecular weight of from about 20,000 to about 50,000 available as MERLON® from Mobay Chemical Company; and a polycarbonate resin having a weight average molecular weight of from about 20,000 to about 80,000 available as PCZ from Mitsubishi Chemicals. Solvents such as methylene chloride, tetrahydrofuran, toluene, monochlorobenzene, or mixtures thereof, may be utilized in forming the charge transport layer coating mixture.

Any suitable charge transporting or electrically active molecules may be employed as HTMs in forming a charge transport layer on a photoreceptor. Suitable charge transporting molecules include, for example, aryl amines as disclosed in U.S. Pat. No. 4,265,990, the entire contents of which are incorporated by reference herein. In embodiments, an aryl amine charge hole transporting component may be represented by:

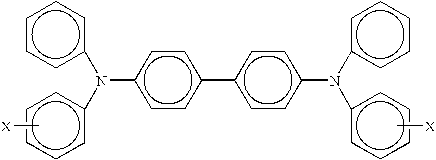

wherein X can be alkyl, halogen, alkoxy or mixtures thereof. In embodiments, the halogen is a chloride. Alkyl groups may contain, for example, from about 1 to about 10 carbon atoms and, in embodiments, from about 1 to about 5 carbon atoms. Examples of suitable aryl amines include, but are not limited to, N,N′-diphenyl-N,N′-bis(alkylphenyl)-1,1-biphenyl-4,4′-diamine, wherein the alkyl may be methyl, ethyl, propyl, butyl, hexyl, and the like; and N,N′-diphenyl-N,N′-bis(halophenyl)-1,1′-biphenyl-4,4′-diamine, wherein the halo may be a chloro, bromo, fluoro, and the like substituent.

Other suitable aryl amines which may be utilized as an HTM in a charge transport layer include, but are not limited to, tritolylamine, N,N′-bis(3,4 dimethylphenyl)-N″(1-biphenyl)amine, 2-bis((4′-methylphenyl)amino-p-phenyl) 1,1-diphenyl ethylene, 1-bisphenyl-diphenylamino-1-propene, triphenylmethane, bis(4-diethylamine-2-methylphenyl)phenylmethane, 4′-4″-bis(diethylamino)-2′,2″-dimethyltriphenylmethane, N,N′-bis(alkylphenyl)-[1,1′-biphenyl]-4,4′-diamine wherein the alkyl is, for example, methyl, ethyl, propyl, n-butyl, etc., N,N′-diphenyl-N,N′-bis(3″-methylphenyl)-(1,1′-biphenyl)-4,4′-diamine, and the like.

The weight ratio of the polymer resin to charge transport molecules in the resulting charge transport layer can be, for example, from about 30/70 to about 80/20. In embodiments the weight ratio of the polymer resin to charge transport molecules can be from about 35/65 to about 75/25, in embodiments from about 40/60 to about 70/30.

Any suitable and conventional technique may be utilized to mix the polymer resin in combination with the hole transport material and apply same as a charge transport layer to a photoreceptor. In embodiments, it may be advantageous to add the polymer resin and hole transport material to a solvent to aid in formation of a charge transport layer and its application to a photoreceptor. Examples of solvents which may be utilized include aromatic hydrocarbons, aliphatic hydrocarbons, halogenated hydrocarbons, ethers, amides and the like, or mixtures thereof. In embodiments, a solvent such as cyclohexanone, cyclohexane, chlorobenzene, carbon tetrachloride, chloroform, methylene chloride, trichloroethylene, toluene, tetrahydrofuran, dioxane, dimethyl formamide, dimethyl acetamide and the like, may be utilized in various amounts. Application techniques of the charge transport layer include spraying, slot or slide coating, dip coating, roll coating, wire wound rod coating, and the like. Drying of the deposited coating may be effected by any suitable conventional technique such as oven drying, infrared radiation drying, air drying and the like.

The thickness of the charge transport layer can be from about 2 micrometers and about 50 micrometers, in embodiments from about 10 micrometers to about 35 micrometers. The charge transport layer should be an insulator to the extent that the electrostatic charge placed on the charge transport layer is not conducted in the absence of illumination at a rate sufficient to prevent formation and retention of an electrostatic latent image thereon. In general, the ratio of the thickness of the charge transport layer to the photogenerating layer, where present, is in embodiments from about 2:1 to 200:1 and in some instances as great as 400:1.

The photogenerating layer, charge transport layer, and other layers may be applied in any suitable order to produce either positive or negative charging photoreceptors. For example, the photogenerating layer may be applied prior to the charge transport layer, as illustrated in U.S. Pat. No. 4,265,990, or the charge transport layer may be applied prior to the photogenerating layer, as illustrated in U.S. Pat. No. 4,346,158, the entire disclosures of each of which are incorporated by reference herein. When used in combination with a charge transport layer, the photogenerating layer may be sandwiched between a conductive surface and a charge transport layer or the charge transport layer may be sandwiched between a conductive surface and a photogenerating layer.

Optionally, an overcoat layer may be applied to the surface of a photoreceptor to improve resistance to abrasion. In some cases, an anti-curl back coating may be applied to the side of the substrate opposite the active layers of the photoreceptor (i.e., the CGL and CTL) to provide flatness and/or abrasion resistance where a web configuration photoreceptor is fabricated. These overcoating and anti-curl back coating layers are known and may include thermoplastic organic polymers or inorganic polymers that are electrically insulating or slightly semi-conductive. For example, overcoat layers may be fabricated from a dispersion including a particulate additive in a resin. Suitable particulate additives for overcoat layers include metal oxides including aluminum oxide, non-metal oxides including silica or low surface energy polytetrafluoroethylene, and combinations thereof. Suitable resins include those described above as suitable for photogenerating layers and/or charge transport layers, for example, polyvinyl acetates, polyvinylbutyrals, polyvinylchlorides, vinylchloride and vinyl acetate copolymers, carboxyl-modified vinyl chloride/vinyl acetate copolymers, hydroxyl-modified vinyl chloride/vinyl acetate copolymers, carboxyl- and hydroxyl-modified vinyl chloride/vinyl acetate copolymers, polyvinyl alcohols, polycarbonates, polyesters, polyurethanes, polystyrenes, polybutadienes, polysulfones, polyarylethers, polyarylsulfones, polyethersulfones, polyethylenes, polypropylenes, polymethylpentenes, polyphenylene sulfides, polysiloxanes, polyacrylates, polyvinyl acetals, polyamides, polyimides, amino resins, phenylene oxide resins, terephthalic acid resins, phenoxy resins, epoxy resins, phenolic resins, polystyrene and acrylonitrile copolymers, poly-N-vinylpyrrolidinones, acrylate copolymers, alkyd resins, cellulosic film formers, poly(amideimide), styrene-butadiene copolymers, vinylidenechloride-vinylchloride copolymers, vinylacetate-vinylidenechloride copolymers, styrene-alkyd resins, polyvinylcarbazoles, and combinations thereof. Overcoatings may be continuous and have a thickness from about 0.5 micrometers to about 10 micrometers, in embodiments from about 2 micrometers to about 6 micrometers.

An example of an anti-curl backing layer is described in U.S. Pat. No. 4,654,284, the entire disclosure of which is incorporated herein by reference. In embodiments, it may be desirable to coat the back of the substrate with an anticurl layer such as, for example, polycarbonate materials commercially available as MAKROLON® from Bayer Material Science. The thickness of anti-curl backing layers should be sufficient to substantially balance the total forces of the layer or layers on the opposite side of the supporting substrate layer. A thickness for an anti-curl backing layer from about 10 micrometers to about 100 micrometers, in embodiments from about 15 micrometers to about 50 micrometers, is a satisfactory range for flexible photoreceptors.

The following Examples are being submitted to illustrate embodiments of the present disclosure. These Examples are intended to be illustrative only and are not intended to limit the scope of the present disclosure. Also, parts and percentages are by weight unless otherwise indicated.

### EXAMPLES

### Example 1

Preparation of high surface area HOGaPc Type I. Ten grams of hydroxygallium phthalocyanine dimer was dissolved in about 100 milliliters of concentrated sulfuric acid at about 50° C. for about 2 hours. The hot solution was allowed to cool down to room temperature, about 23° C. to about 25° C. The cool solution was filtered through a 5-micron glass filter. The resulting solution was subsequently quenched with a mixture of about 400 milliliter ammonia/about 250 milliliter water/about 250 milliliter THF and cooled by dry ice to a temperature of about −10° C. The quenching took about 45 minutes, after which the mixture was stirred for an additional 45 minutes. The mixture was then filtered under vacuum suction, and washed with hot water (>90° C.) twice, and cold water (at room temperature) once, until the filtrate showed a conductivity of from about 0 μS to about 100 μS. (The conductivity was obtained using an OAKTON CON5 ACORN Series conductivity meter.) The pigment was then dried under vacuum at about 65° C. overnight for about 12 to about 20 hours.

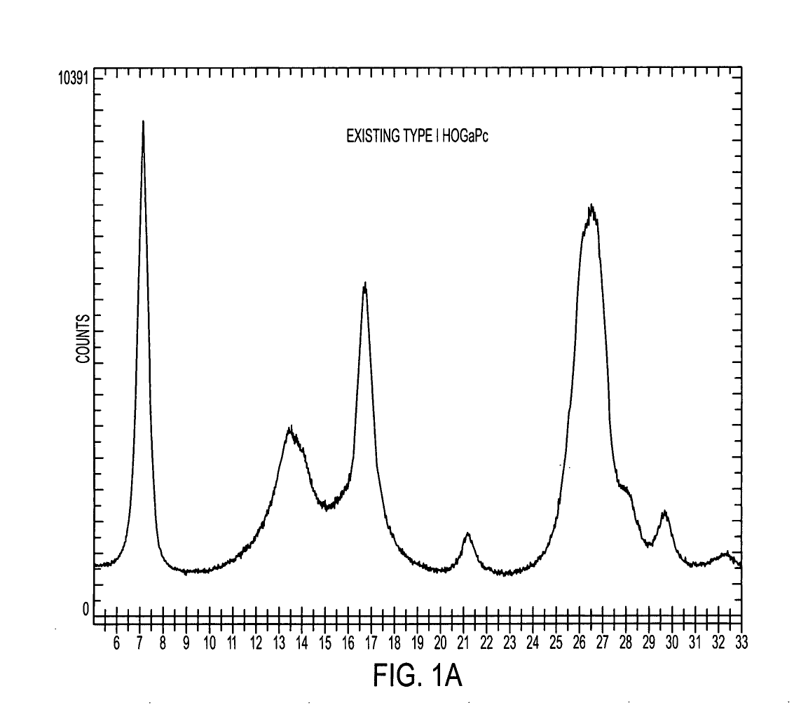

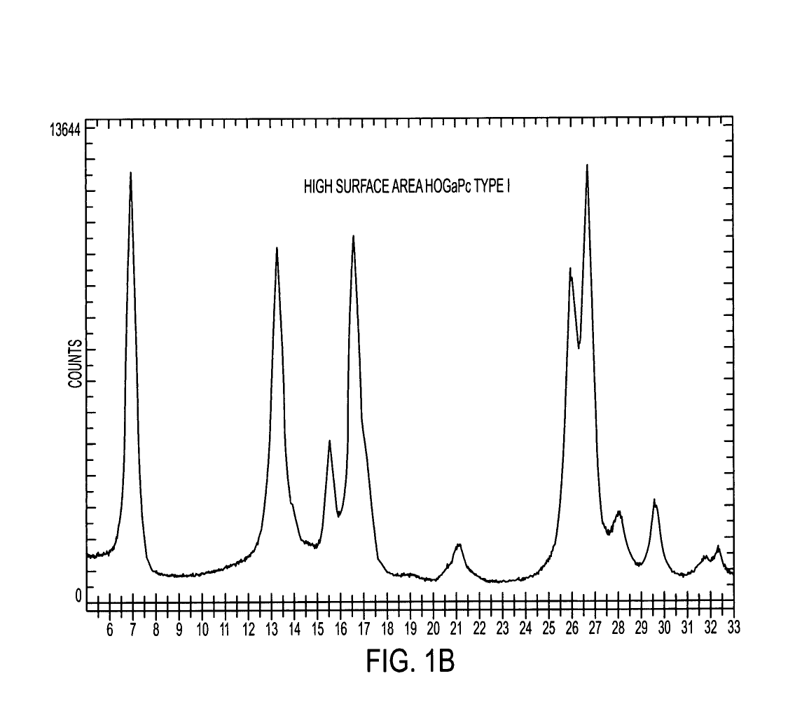

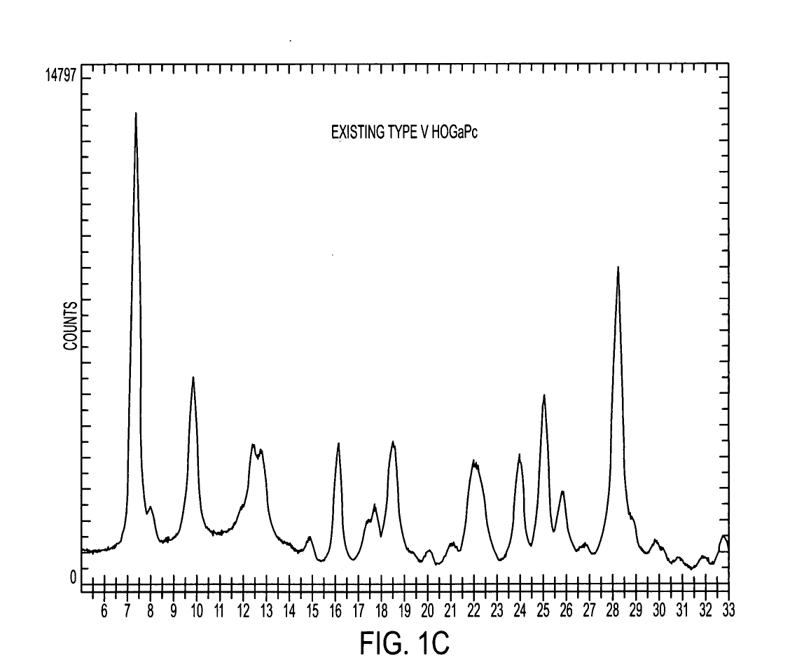

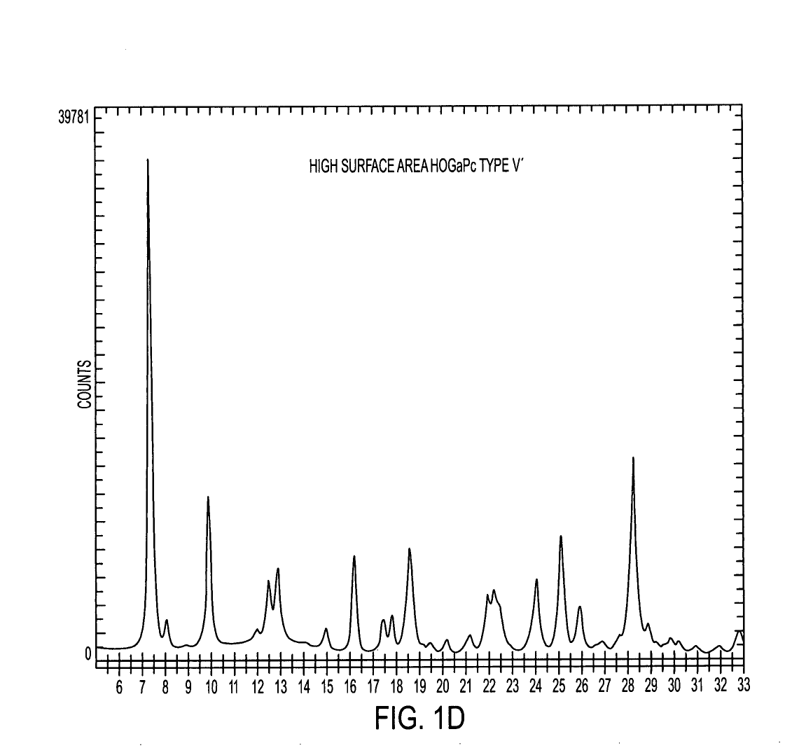

The X-ray diffraction (XRD) of the resulting Type I HOGaPc was obtained using a Siemens D5000 x-ray diffractometer and compared with a control HOGaPc Type I obtained by acid pasting in aqueous ammonium solution without THF. The XRD results are set forth in FIG. 1.

### Example 2

Conversion of high surface area HOGaPc Type I to Type V. About 3 grams of high surface area HOGaPc Type I obtained in Example 1 above was mixed with about 30 grams of DMF and about 100 grams of 1 mm HiBea borosilicate glass beads (Ohara Inc., Kanagawa, Japan) in a 120 ml bottle. The bottle was rolled at about 60 rpm for about 5 days on a roll mill. After rolling, the beads were removed from the pigment slurry, and the slurry was then filtered to collect high surface area Type V pigment. The pigment was then washed with acetone, and dried at about 85° C. under vacuum.

The X-ray diffraction (XRD) for the high surface area HOGaPc Type V pigment was obtained using a Siemens D5000 x-ray diffractometer and compared with HOGaPc Type V obtained from the conversion of the control HOGaPc Type I in Example 1 above (control HOGaPc Type V). The XRD results are set forth in FIG. 1.

### Example 3

Preparation of high surface area HOGaPc Type V charge generating coating dispersion. About 2.5 grams of high surface area HOGaPc Type V pigment prepared in Example 2 above was mixed with about 1.67 grams of poly(vinyl chloride/vinyl acetate) copolymer (VMCH from Dow Chemical) and about 30 grams of n-butyl acetate. The mixture was milled in an Attritor mill with about 130 grams of 1 mm Hi-Bea borosilicate glass beads for about 1.5 hours. The dispersion was filtered through a 20-μm nylon cloth filter, and the solid content of the dispersion was diluted to about 5 weight percent with n-butyl acetate.

As a control, the same procedures were utilized to prepare a charge generating layer dispersion having control HOGaPc Type V produced in Example 2 above as the pigment.

Several photoreceptor devices were prepared to compare the various electrical properties of the high surface area HOGaPc Type V pigment of the present disclosure with the control HOGaPc Type V pigment produced in Example 2 above. The photoreceptor devices included an undercoat layer, a charge generating layer made of the high surface area HOGaPc Type V dispersion (Device 1) or control HOGaPc Type V dispersion (Device 2), and a charge transport layer.

All the devices possessed a 3-component undercoat layer, varying HOGaPc charge generating layer, and about an 18-μm thick charge transport layer. The 3-component undercoat layer was prepared as follows: zirconium acetylacetonate tributoxide (about 35.5 parts), γ-aminopropyltriethoxysilane (about 4.8 parts) and poly(vinyl butyral) (about 2.5 parts) were dissolved in n-butanol (about 52.2 parts) to prepare a coating solution. The coating solution was coated via a ring coater, and the layer was pre-heated at about 59° C. for about 13 minutes, humidified at about 58° C. (dew point of 54° C.) for about 17 minutes, and then dried at about 135° C. for about 8 minutes. The thickness of the undercoat layer on each photoreceptor was approximately 1.3 μm. The HOGaPc photogenerating layer dispersions were prepared as described above, and applied on top of the 3-component undercoat layer. The thickness of the photogenerating layer was approximately 0.2 μm for each photoreceptor; the thickness was controlled by pull rate (the higher the pull rate, the thicker the photogenerating coating). Subsequently, an 18-μm charge transport layer (CTL) was coated on top of the photogenerating layer from a solution of N,N′-diphenyl-N,N-bis(3-methyl phenyl)-1,1′-biphenyl-4,4′-diamine (about 9.9 grams) and a polycarbonate, PCZ-400 [poly(4,4′-dihydroxy-diphenyl-1-1-cyclohexane, Mw=40000)] available from Mitsubishi Gas Chemical Co., Ltd. (about 12.1 grams), in a mixture of about 55 grams of tetrahydrofuran (THF) and about 23.5 grams of monochlorobenzene. The charge transport layer was dried at about 135° C. for about 45 minutes.

The above prepared photoreceptor devices were tested in a scanner set to obtain photo induced discharge cycles, sequenced at one charge-erase cycle followed by one charge-expose-erase cycle, wherein the light intensity was incrementally increased with cycling to produce a series of photo induced discharge characteristic curves (PIDC) from which the photosensitivity and surface potentials at various exposure intensities were measured. Additional electrical characteristics were obtained by a series of charge-erase cycles with incrementing surface potential to generate several voltages versus charge density curves. The scanner was equipped with a scorotron set to a constant voltage charging at various surface potentials. The devices were tested at surface potentials of about 500 and about 700 volts with the exposure light intensity incrementally increased by means of regulating a series of neutral density filters; the exposure light source was a 780-nanometer light emitting diode. The aluminum drum was rotated at a speed of about 55 revolutions per minute to produce a surface speed of about 277 millimeters per second or a cycle time of about 1.09 seconds. The xerographic simulation was completed in an environmentally controlled light tight chamber at ambient conditions (about 40 percent relative humidity and about 22° C.). The following properties of the photoreceptors were obtained as follows: sensitivity (S) was measured (in units of volt cm2/ergs) as the initial slope of a photoinduced discharge characteristic (PIDC) curve; V_{depl}, a measurement of voltage leak during charging, was linearly extrapolated from the surface potential versus charge density relation of the device; and dark decay (V_{dd}) was the lost potential before light exposure. An excellent photoreceptor device should have V_{dd} and V_{depl} close to zero. The results are set forth in Table 1 below.

TABLE 1

| | S (Vcm2/erg) | V_{depl} (V) | V_{dd} (V) |
|---|---|---|---|
| Device 1 | −242 | 45 | 16 |
| Device 2 | −238 | 44 | 18 |

Almost identical PIDC characteristics were observed from the device with the high surface area HOGaPc Type V pigment of the present disclosure (Device 1) and the device with the control HOGaPc Type V pigment (Device 2).

The print quality, in particular background (at about 52 mm/s process speed) under A zone (30° C./80% humidity), for each photoreceptor was evaluated by an IMARI printer manufactured by Xerox, and graded on a scale of 1 to 7 by visual inspection for background shading. The results of this testing are summarized in Table 2 below, where the lower the A zone background, the better the print quality.

TABLE 2

| | A zone background (grade 1 to 7) |
|---|---|
| Device 1 | 3 |
| Device 2 | 6.5 |

As is apparent from Table 2 above, photoreceptors having a high surface area HOGaPc Type V pigment of the present disclosure (Device 1) had much better print quality compared with a photoreceptor having the control HOGaPc Type V pigment.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims.

What is claimed is:

1. A process comprising contacting a gallium phthalocyanine in an acid solution with a solvent system comprising water, at least one base, and at least one water miscible solvent.

2. The process of claim 1 wherein the gallium phthalocyanine in acid solution and solvent system is at a temperature from about −20° C. to about 40° C., the at least one base comprises from about 2 to about 5 bases, and the at least one water miscible solvent comprises from about 2 to about 5 solvents.

3. The process of claim 1 wherein the gallium phthalocyanine comprises a halogallium phthalocyanine.

4. The process of claim 1 wherein the gallium phthalocyanine comprises a chlorogallium phthalocyanine.

5. The process of claim 1 wherein the gallium phthalocyanine comprises an alkoxy-bridged phthalocyanine dimer.

6. The process of claim 1 wherein the base has a pKb from about 1 to about 10, the water miscible solvent is selected from the group consisting of cyclic ethers, amides, polyols, nitrites, sulfur containing solvents, and mixtures thereof, and wherein the amount of base in the solvent system is from about 10 percent by volume to about 70 percent by volume and the water miscible solvent in the solvent system is from about 5 to about 70 volume percent based upon total components of the solvent system.

7. The process of claim 1 wherein the base is selected from the group consisting of an aqueous ammonia solution, ammonia, trimethylammonia, pyridine, hydrazine, hydroxylamine, methylamine, ethylamine, dimethylamine, diethylamine, ethanolamine, triethanolamine, ethyldiamine, urea, aqueous hydrogen bisulfide solution, and aqueous formate solution, the water miscible solvent is selected from the group consisting of tetrahydrofuran, N,N-dimethylformamide, N,N-dimethylacetamide, dimethyl sulfoxide, tetramethylene sulfone, acetonitrile, 1,3-dioxane, 1,4-dioxane, ethylene glycol, 1-methyl-2-pyrrolidinone, and combinations thereof, and wherein the amount of base in the solvent system is from about 30 percent by volume to about 50 percent by volume and the water miscible solvent in the solvent system is from about 20 to about 40 volume percent based upon the total components of the solvent system.

8. The process of claim 1 wherein the solvent system comprises ammonia, deionized water, and at least one water miscible solvent selected from the group consisting of tetrahydrofuran, N,N-dimethylformamide, and mixtures thereof, and wherein the process forms particles comprising a Type I hydroxygallium phthalocyanine having a surface area of from about 50 m²/g to about 90 m²/g.

9. The process of claim 8 wherein particles comprising the Type I hydroxygallium phthalocyanine have a surface area of from about 60 m²/g to about 70 m²/g.

10. The process of claim 8 further comprising converting the Type I hydroxygallium phthalocyanine to a Type V hydroxygallium phthalocyanine by contacting the Type I hydroxygallium phthalocyanine with a polar aprotic solvent, and wherein particles comprising the Type V hydroxygallium phthalocyanine have a surface area of from about 50 m²/g to about 100 m²/g.

11. The process of claim 10 wherein particles comprising the Type V hydroxygallium phthalocyanine have a surface area of from about 60 m²/g to about 80 m²/g, and wherein the Type V hydroxygallium phthalocyanine has major peaks at Bragg angles (2 theta±0.2°) of 7.5°, 9.9°, 12.5°, 16.3°, 18.6°, 21.9°, 23.9°, 25.1°, 28.3° and with the highest peak at 7.5 degrees.

12. A process comprising contacting an alkoxy-bridged gallium phthalocyanine dimer in an acid solution with a solvent system comprising water, aqueous ammonia solution, and at least one water miscible solvent selected from the group consisting of cyclic ethers, amides, polyols, nitriles, sulfur-containing solvents, and mixtures thereof to obtain a Type I hydroxygallium phthalocyanine, and converting said Type I hydroxygallium phthalocyanine to a Type V hydroxygallium phthalocyanine, wherein particles comprising the Type V hydroxygallium phthalocyanine have a surface area of from about 50 m²/g to about 100 m²/g.

13. The process of claim 12 wherein the aqueous ammonia solution contains from about 28 to about 30 weight percent of ammonia, wherein the at least one water miscible solvent is selected from the group consisting of tetrahydrofuran, N,N-dimethylformamide, N,N-dimethylacetamide, dimethyl sulfoxide, tetramethylene sulfone, acetonitrile, 1,3-dioxane, 1,4-dioxane, ethylene glycol, 1-methyl-2-pyrrolidinone, and combinations thereof, and wherein the at least one water miscible solvent is present in the solvent system from about 5 to about 70 volume percent based upon the total components of the solvent system.

14. The process of claim 12 wherein the at least one water miscible solvent is present in the solvent system from about 20 to about 40 volume percent based upon the total components of the solvent system, wherein particles comprising the Type V hydroxygallium phthalocyanine have a surface area of from about 60 m²/g to about 80 m²/g, and wherein the Type V hydroxygallium phthalocyanine has major peaks at Bragg angles (2 theta±0.2°) of 7.5°, 9.9°, 12.5°, 16.3°, 18.6°, 21.9°, 23.9°, 25.1°, 28.3° and with the highest peak at 7.5 degrees.

15. A photoreceptor comprising a photogenerating layer comprising a resin and a photogenerating component comprising a hydroxygallium phthalocyanine prepared by contacting a gallium phthalocyanine in an acid solution with a solvent system comprising water, aqueous ammonia solution, and at least one water miscible solvent to obtain a Type I hydroxygallium phthalocyanine, and converting said Type I hydroxygallium phthalocyanine to a Type V hydroxygallium phthalocyanine.

16. The photoreceptor of claim 15 wherein the photogenerating layer comprises about 15 weight percent to about 95 weight percent of the resin and about 5 weight percent to about 85 weight percent of the photogenerating component, and wherein particles comprising the Type V hydroxygallium phthalocyanine have a surface area of from about 60 m²/g to about 80 m²/g.

17. The photoreceptor of claim 15 wherein the gallium phthalocyanine is selected from the group consisting of halogallium phthalocyanines and alkoxy-bridged gallium phthalocyanines, the at least one water miscible solvent comprises from about 2 to about 5 solvents, the at least one water miscible solvent is selected from the group consisting of tetrahydrofuran, N,N-dimethylformamide, N,N-dimethylacetamide, dimethyl sulfoxide, tetramethylene sulfone, acetonitrile, 1,3-dioxane, 1,4-dioxane, ethylene glycol, 1-methyl-2-pyrrolidinone, and combinations thereof, and the Type I hydroxygallium phthalocyanine is converted to the Type V hydroxygallium phthalocyanine by contacting the Type I hydroxygallium phthalocyanine with a polar aprotic solvent.

18. The photoreceptor of claim 15 wherein the Type V hydroxygallium phthalocyanine has major peaks at Bragg angles (2 theta±0.2°) of 7.5°, 9.9°, 12.5°, 16.3°, 18.6°, 21.9°, 23.9°, 25.1°, 28.3° and with the highest peak at 7.5 degrees.

19. The photoreceptor of claim 15, further comprising a charge transport layer comprising hole transport molecules comprising aryl amines, an optional substrate, an optional hole blocking layer, and an optional adhesive layer, and wherein the thickness of the photogenerating layer is from about 0.05 microns to about 10 microns and the thickness of the charge transport layer is from about 2 micrometers to about 50 micrometers.

20. The photoreceptor of claim 19, wherein the charge transport layer comprises hole transport molecules comprising an aryl amine of the formula 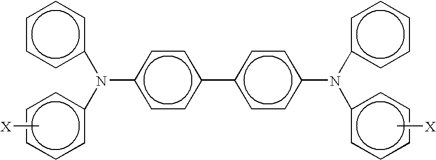
wherein X is selected from the group consisting of alkyl, halogen, alkoxy or mixtures thereof.
